(19) United States
(12) Patent Application Publication
(10) Pub. No.: US 2024/0067416 A1
(43) Pub. Date: Feb. 29, 2024

(54) NOVEL CUP LID STRUCTURE OF FRAGRANCE CUP

(71) Applicant: CROWN (Yangzhou) HEALTH & Beauty Co., Ltd., Yangzhou (CN)

(72) Inventor: Wenxiang Li, Yangzhou (CN)

(21) Appl. No.: 18/120,510

(22) Filed: Mar. 13, 2023

(30) Foreign Application Priority Data
Aug. 27, 2022 (CN) 202222265695.9

(51) Int. Cl. B65D 47/06; A61L 9/12; B65D 51/28

(52) U.S. Cl. CPC B65D 47/06; A61L 9/125; B65D 51/28; B65D 2543/00046

(57) ABSTRACT

The present utility model provides a novel cup lid structure of a fragrance cup, comprising a cup lid body and a suction nozzle installed on the cup lid body, wherein a flavor packet accommodating part which can rotate relative to the center of the suction nozzle is installed at the outer diameter of the suction nozzle, and a plurality of cavities for placing flavor packets are arranged in the flavor packet accommodating part; the cup lid further comprises a channel which enables the single cavity to communicate with the central hole of the suction nozzle and the outside air when the flavor packet accommodating part rotates to a designated position. The present utility model is simple in design and structure, and convenient to operate, and can put flavor packets with different scents in the flavor packet accommodating part. By rotating the flavor packet accommodating part, users can experience different scents when drinking water, so as to meet the use requirements of different users. In addition, the check valve is arranged in the suction nozzle, which can avoid the problem of water spilling when the water cup is tilted and improve the use experience.

### TECHNICAL FIELD

The present utility model relates to the technical field of cup design, in particular to a novel cup lid structure of a fragrance cup.

### BACKGROUND

It is found that if you smell the fruit aroma or flower fragrance when drinking white water, the water you drink into your mouth seems to have the fruit aroma or flower fragrance, which is the embodiment of the intercommunication of smell and flavor.

The healthy diet consultant advocates that everyone should always drink more water to ensure the metabolism in the body, promote the discharge of harmful substances and ensure the health of the body; however, the flavor of boiled water and purified water is too light, so many people can't insist on drinking more water every day, the basic amount of drinking water can't be guaranteed, and the health condition can't be improved. Therefore, in the prior art, a fragrance cup is used to increase the fragrance flavor when drinking water, thereby improving the flavor. However, the existing fragrance cup can only provide one fragrance, and can't choose different flavors according to actual needs, which has the disadvantage of small application range; Moreover, the suction nozzle of the existing water cup is directly communicated with the water in the cup, which causes the problem of water spilling when the water cup is tilted and is inconvenient to use.

### SUMMARY

The present utility model provides a novel cup lid structure of a fragrance cup, in order to solve the problems that the existing fragrance cup can only provide one flavor, can not be selected according to the user's needs, has a small application range, and is easy to spill water when the cup is tilted.

The technical solution of the present utility model is realized as follows:
a novel cup lid structure of a fragrance cup, including a cup lid body and a suction nozzle installed on the cup lid body, wherein a flavor packet accommodating part which can rotate relative to a center of the suction nozzle is installed at an outer diameter of the suction nozzle, and a plurality of cavities or placing flavor packets are arranged in the flavor packet accommodating part;
the cup lid structure further comprises a channel that enables the single cavity to communicate with a central hole of the suction nozzle and outside air when the flavor packet accommodating part rotates to a designated position.

Preferably, the flavor packet accommodating part comprises a bottom shell and an upper lid detachably connected above the bottom shell, wherein a middle part of the bottom shell is provided with an annular groove coaxial with the bottom shell, and a plurality of clapboards are fixedly arranged in the annular groove; the clapboards evenly divide the annular groove into a plurality of cavities.

Preferably, the channel comprises a bottom hole opened at a bottom of the cavity, a sidewall hole opened at an inner sidewall of the cavity, a vent hole opened at a side of the suction nozzle, and an L-shaped groove opened on the cup lid body; a center of the sidewall hole is set at a same height as a center of the vent hole; when the flavor packet accommodating part rotates to a specified position, the vent hole, the sidewall hole, the cavity and the bottom hole are communicated with each other, and are communicated at a channel formed by the L-shaped groove and the flavor packet accommodating part.

Preferably, both edge positions of the bottom shell and the upper lid are provided with anti-skid protrusions.

Preferably, an upper edge of the bottom shell is provided with a positioning table extending outward, and an inner wall of the upper lid is provided with a positioning groove corresponding to the positioning table.

Preferably, a lower arc-shaped groove with a semicircular cross-section is provided at a top of the inner sidewall of the cavity of the bottom shell, and an upper arc-shaped groove corresponding to the lower arc-shaped groove is provided on a lower surface of the upper lid; after the upper lid and the bottom shell are assembled, the lower arc-shaped groove and the upper arc-shaped groove form the sidewall hole.

Preferably, an outer diameter of a lower end of the suction nozzle is provided with a limit ring protruding outwards, the limit ring passes through a central hole of the cup lid body and axially limits the suction nozzle through the limit ring; an outer wall of the suction nozzle is provided with a clamping groove, and an inner wall of the cup lid body is provided with a limit table extending in a center direction of the cup lid body, and the limit table is placed at the clamping groove.

Preferably, a check valve is installed at the central hole of the suction nozzle.

By adopting the technical solution, the present utility model has the beneficial effects that:

The present utility model is simple in design and structure, and convenient to operate, and can put flavor packets with different scents in the flavor packet accommodating part. By rotating the flavor packet accommodating part, users can experience different scents when drinking water, so as to meet the use requirements of different users. In addition, the check valve is arranged in the suction nozzle, which can avoid the problem of water spilling when the water cup is tilted and improve the use experience.

### BRIEF DESCRIPTION OF DRAWINGS

In order to explain the embodiments of the present utility model or the technical solution in the prior art more clearly, the drawings needed in the description of the embodiments or the prior art will be briefly introduced below. Obviously, the drawings in the following description are only some embodiments of the present utility model. For those skilled in the art, other drawings can be obtained according to these drawings without paying creative labor.

FIG. 1 is a perspective view of the present utility model;

FIG. 2 is an exploded view of the present utility model;

FIG. 3 is a sectional view of the present utility model;

FIG. 4 is a perspective view of the cup lid body of the present utility model;

FIG. 5 is a perspective view of the suction nozzle of the present utility model;

FIG. 6 is a sectional view of the suction nozzle of the present utility model;

FIG. 7 is a perspective view of the bottom shell of the present utility model;

FIG. 8 is a perspective view of the upper lid of the present utility model;

FIG. 9 is a partial enlarged view of FIG. 6 of the present utility model.

In the figures:
1, Cup lid body; 101, L-shaped groove; 102, Limit table; 2, Suction nozzle; 201, Vent hole; 202, Limit ring; 203, Clamping groove; 204, Blocking step; 3, Flavor packet accommodating part; 301, Bottom shell; 3011, Clapboard; 3012, Lower arc-shaped groove; 3013, Bottom hole; 3014, Positioning table; 302, Upper lid; 3021, Upper arc-shaped groove; 3022, Positioning groove; 303, Cavity; 304, Sidewall hole; 4, Check valve; 5, Anti-slip protrusion.

### DESCRIPTION OF EMBODIMENTS

The technical solution in the embodiment of this utility model will be described clearly and completely with the attached drawings. Obviously, the described embodiment is only a part of the embodiment of this utility model, but not the whole embodiment. Based on the embodiments in this utility model, all other embodiments obtained by those skilled in the art without creative work belong to the protection scope of this utility model.

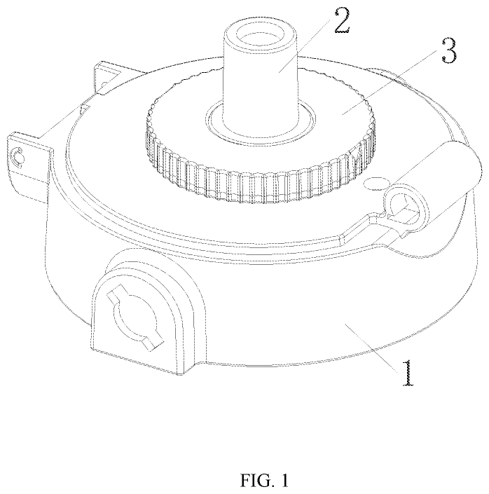

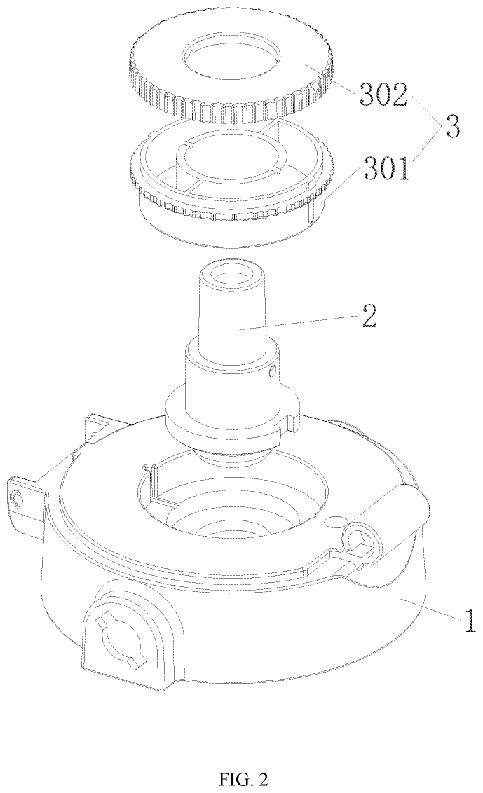

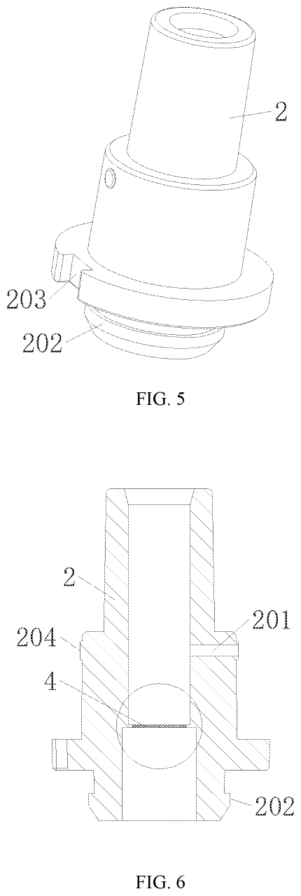

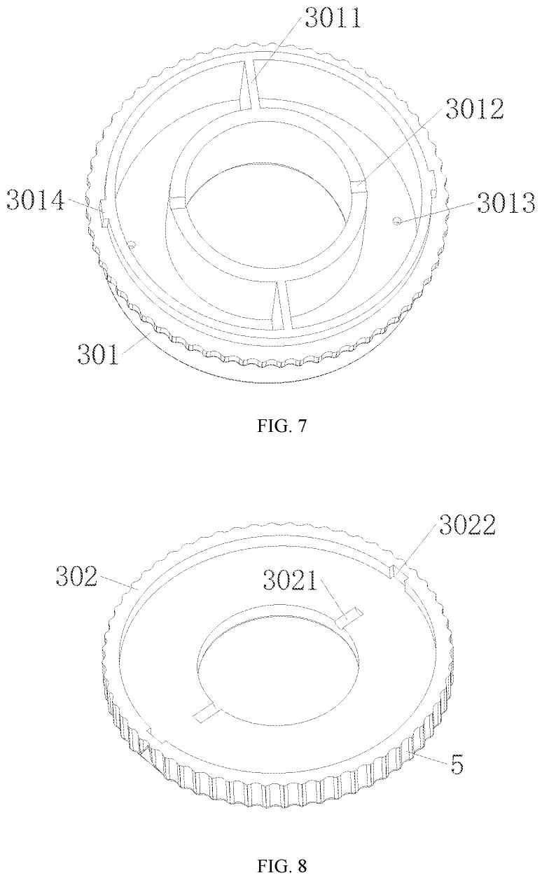

Referring to FIGS. 1-9, a novel cup lid structure of a fragrance cup includes a cup lid body 1 and a suction nozzle 2 installed on the cup lid body 1, wherein a flavor packet accommodating part 3 which can rotate relative to a center of the suction nozzle 2 is installed at an outer diameter of the suction nozzle 2, and a plurality of cavities 303 for placing flavor packets are arranged in the flavor packet accommodating part 3;
the cup lid structure further comprises a channel that enables the single cavity 303 to communicate with a central hole of the suction nozzle 2 and outside air when the flavor packet accommodating part 3 rotates to a designated position.

Specifically, the flavor packet accommodating part 3 incudes a bottom shell 301 and an upper lid 302 detachably connected above the bottom shell 301, wherein a middle part of the bottom shell 301 is provided with an annular groove coaxial with the bottom shell 301, and a plurality of clapboards 3011 are fixedly arranged in the annular groove; the clapboards 3011 evenly divide the annular groove into a plurality of cavities 303. The purpose of the upper lid 302 is to facilitate the opening, so as to put the flavor packet into different cavities 303.

Specifically, the channel includes a bottom hole 3013 opened at a bottom of the cavity 303, a sidewall hole 304 opened at an inner sidewall of the cavity 303, a vent hole 201 opened at a side of the suction nozzle 2, and an L-shaped groove 101 opened on the cup lid body 1; a center of the sidewall hole 304 is set at a same height as a center of the vent hole 201; when the flavor packet accommodating part 3 rotates to a specified position, the vent hole 201, the sidewall hole 304, the cavity 303 and the bottom hole 3013 are communicated with each other, and are communicated at a channel formed by the L-shaped groove 101 and the flavor packet accommodating part 3. This arrangement is made so that only the fragrance in one cavity 303 can enter the suction nozzle 2 at one time. In addition, a protruding blocking step 204 is provided on the outer wall of the suction nozzle 2, so that the channels formed by the other cavities 303 are blocked by the blocking step 204 when one of the channels is kept unobstructed.

Specifically, the edge positions of the bottom shell 301 and the upper lid 302 are both provided with anti-skid protrusions 5 for increasing the friction with hands and facilitating the rotation of the bottom shell 301 and the upper lid 302. More specifically, the upper surface of the cup lid body 1 has an indicator arrow pointing in the direction of the L-shaped groove 101, and in addition, there is also an indicator arrow on the upper lid 302, and the indicator arrow of the upper lid 302 points to the position of the upper arc-shaped groove 3021.

Specifically, the upper edge of the bottom shell 301 has a positioning table 3014 extending outward, and the inner wall of the upper lid 302 is provided with a positioning groove 3022 corresponding to the positioning table 3014, so that the bottom shell 301 can rotate together when the upper lid 302 is rotated.

Specifically, a lower arc-shaped groove 3012 with a semicircular cross-section is provided at a top of the inner sidewall of the cavity 303 of the bottom shell 301, and an upper arc-shaped groove 3021 corresponding to the lower arc-shaped groove 3012 is provided on a lower surface of the upper lid 302; after the upper lid 302 and the bottom shell 301 are assembled, the lower arc-shaped groove 3012 and the upper arc-shaped groove 3021 form the sidewall hole 304. This design is intended to facilitate the cleaning of the sidewall hole 304.

Specifically, an outer diameter of a lower end of the suction nozzle 2 is provided with a limit ring 202 protruding outwards, the limit ring 202 passes through a central hole of the cup lid body 1 and axially limits the suction nozzle 2 through the limit ring 202; an outer wall of the suction nozzle 2 is provided with a clamping groove 203, and an inner wall of the cup lid body 1 is provided with a limit table 102 extending in a center direction of the cup lid body 1, and the limit table 102 is placed at the clamping groove 203. The suction nozzle 2 is made of silica gel. The purpose of setting the limit ring 202, the clamping groove 203 and the limit table 102 is to keep the suction nozzle 2 and the lid body 1 relatively static, and to prevent the suction nozzle 2 from rotating when the flavor packet accommodating part 3 rotates.

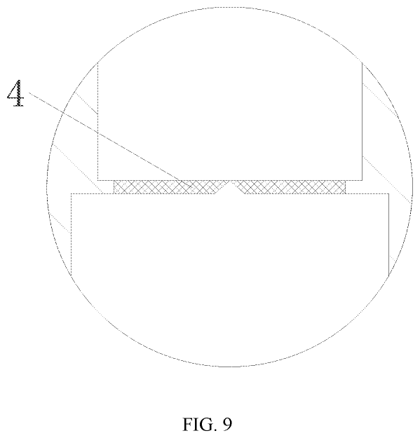

Specifically, a check valve 4 is installed at the central hole of the suction nozzle 2, which is mainly used to prevent water from spilling when the water cup is tilted. More specifically, as shown in FIG. 9, the check valve 4 is made of silica gel and has two openable and closable blades. When drinking water, the blades are opened, and the water can move upward through the gap opened by the check valve 4 under the suction force. When idle, the blades are closed, thus preventing water from spilling.

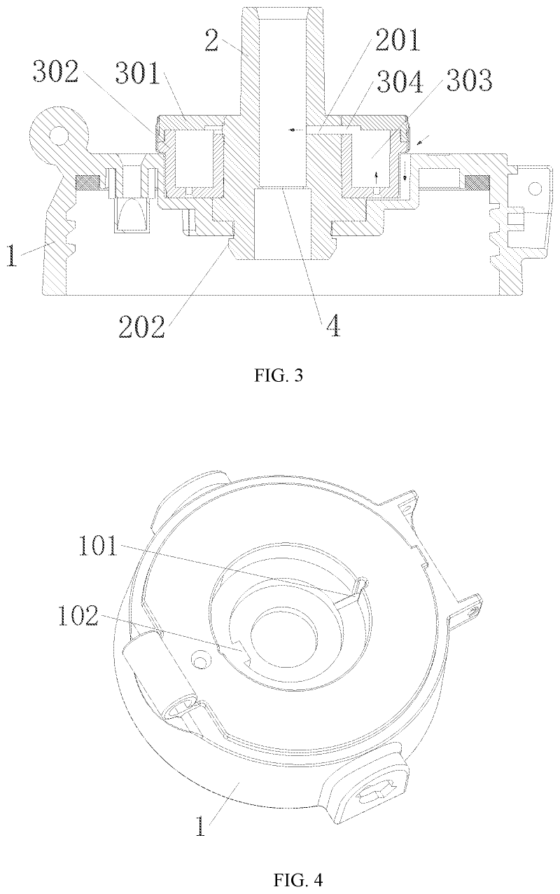

Working principle: when in use, flavor packets with different flavors can be placed in the cavity 303. When drinking water, the cavity 303 where one flavor packet is located can be communicated with the vent hole 201 by rotating the flavor packet accommodating part 3. At the same time, the bottom hole 3013 at the bottom of the cavity 303 is rotated to the position where the L-shaped groove 101 is located, so as to realize the communication between the cavity 303 and the outside air. During drinking water, under the action of suction, the outside air enters the bottom hole 3013 from the channel where the L-shaped groove 101 is located, then enters the cavity 303 where the flavor packet is located through the bottom hole 3013, enters the central hole of the suction nozzle 2 through the sidewall hole 304 and the vent hole 201, and finally enters the human mouth, as shown by the arrow in FIG. 3. When it is necessary to select flavor packets with different flavors, it is convenient and quick to select different flavors only by rotating the flavor packet accommodating part 3.

The above is only the preferred embodiment of this utility model, and it is not used to limit this utility model. Any modification, equivalent substitution, improvement, etc. made within the spirit and principle of this utility model should be included in the protection scope of this utility model.

What is claimed is:

1. A novel cup lid structure of a fragrance cup, comprising a cup lid body and a suction nozzle installed on the cup lid body, wherein a flavor packet accommodating part which can rotate relative to a center of the suction nozzle is installed at an outer diameter of the suction nozzle, and a plurality of cavities for placing flavor packets are arranged in the flavor packet accommodating part;
the cup lid structure further comprises a channel that enables the single cavity to communicate with a central hole of the suction nozzle and outside air when the flavor packet accommodating part rotates to a designated position.

2. The novel cup lid structure of a fragrance cup according to claim 1, wherein the flavor packet accommodating part comprises a bottom shell and an upper lid detachably connected above the bottom shell, wherein a middle part of the bottom shell is provided with an annular groove coaxial with the bottom shell, and a plurality of clapboards are fixedly arranged in the annular groove; the clapboards evenly divide the annular groove into a plurality of cavities.

3. The novel cup lid structure of a fragrance cup according to claim 1, wherein the channel comprises a bottom hole opened at a bottom of the cavity, a sidewall hole opened at an inner sidewall of the cavity, a vent hole opened at a side of the suction nozzle, and an L-shaped groove opened on the cup lid body; a center of the sidewall hole is set at a same height as a center of the vent hole; when the flavor packet accommodating part rotates to a specified position, the vent hole, the sidewall hole, the cavity and the bottom hole are communicated with each other, and are communicated at a channel formed by the L-shaped groove and the flavor packet accommodating part.

4. The novel cup lid structure of a fragrance cup according to claim 2, wherein both edge positions of the bottom shell and the upper lid are provided with anti-skid protrusions.

5. The novel cup lid structure of a fragrance cup according to claim 2, wherein an upper edge of the bottom shell is provided with a positioning table extending outward, and an inner wall of the upper lid is provided with a positioning groove corresponding to the positioning table.

6. The novel cup lid structure of a fragrance cup according to claim 2, wherein a lower arc-shaped groove with a semicircular cross-section is provided at a top of the inner sidewall of the cavity of the bottom shell, and an upper arc-shaped groove corresponding to the lower arc-shaped groove is provided on a lower surface of the upper lid; after the upper lid and the bottom shell are assembled, the lower arc-shaped groove and the upper arc-shaped groove form the sidewall hole.

7. The novel cup lid structure of a fragrance cup according to claim 1, wherein an outer diameter of a lower end of the suction nozzle is provided with a limit ring protruding outwards, the limit ring passes through a central hole of the cup lid body and axially limits the suction nozzle through the limit ring; an outer wall of the suction nozzle is provided with a clamping groove, and an inner wall of the cup lid body is provided with a limit table extending in a center direction of the cup lid body, and the limit table is placed at the clamping groove.

8. The novel cup lid structure of a fragrance cup according to claim 1, wherein a check valve is installed at the central hole of the suction nozzle.
